(19) United States
(12) Patent Application Publication
(10) Pub. No.: US 2023/0360747 A1
(43) Pub. Date: Nov. 9, 2023

(54) IMPROVEMENTS IN OR RELATING TO QUANTITATIVE ANALYSIS OF SAMPLES

(71) Applicant: Fluidic Analytics Limited, Cambridge Cambridgeshire (GB)

(72) Inventors: Alekszej Morgunov, Tallinn (EE); Simon Morling, Cambridge (GB)

(73) Assignee: Fluidic Analytics Limited, Cambridge Cambridgeshire (GB)

(21) Appl. No.: 18/026,385

(22) PCT Filed: Sep. 16, 2021
(86) PCT No.: PCT/GB2021/052400

§ 371 Date: Mar. 15, 2023

(30) Foreign Application Priority Data
Sep. 16, 2020 (GB) 2014608.0

(51) Int. Cl. G16H 10/40; G06N 20/00; G16H 10/60; G16H 50/20; G16H 50/70; G01N 33/68

(52) U.S. Cl. CPC G16H 10/40; G06N 20/00; G16H 10/60; G16H 50/20; G16H 50/70; G01N 33/6803

(57) ABSTRACT

A system is provided for improving the quantitative analysis of a sample. The system comprises a device; a data store and processing circuitry configured to operate the system. The device is configured to perform quantitative analysis of bio-macromolecular interactions in solution on a fluid sample to provided quantitative analysis data. The data store stores: personal data relating to a plurality of individuals; and data relating to bio-macromolecular interactions. The processing circuitry is configured to access the data store and identify and retrieve data relevant to the sample; set the parameters under which the quantitative analysis of the sample is performed in the device in dependence upon said retrieved data; perform analysis using a general model to create a predicted result of the quantitative analysis from the device; receive quantitative analysis data of the sample from the device; compare said quantitative analysis received from the device with the predicted result; and update said data store with at least one of the output of the comparison and said received

The present invention relates to the quantitative analysis of samples and, in particular, to improvements in the intelligence gathering and knowledge processing leading to improved experimental design.

Diagnostics has historically been a substantially binary field, identifying the presence or absence of a disease, the presence or absence of immunity etc. However, the field of biomedical data is multidimensional and complex and it is increasingly the case that a binary output is insufficient to capture the complexity and nuance of the system.

In recent times, many have studied the interactions between biomolecules such as proteins to help develop more sophisticated diagnostics/medical tools for various diseases. Protein-protein interactions (PPIs) form the basis of many biologically and physiologically relevant processes including: protein self-assembly; protein-aggregation; antibody-antigen recognition; muscle contraction and cellular communication. Nevertheless, studying protein-protein interactions, especially under physiological conditions in complex media, remains challenging. Current techniques, such as an enzyme-linked immunosorbent assay (ELISA) bead-based multiplex assay and surface plasmon resonance (SPR) spectroscopy, rely on immobilisation of one binding partner. These techniques include potential unspecific interactions with the surface, which can cause false positive results and the Hook/Prozone effect, which causes false-negative results, thereby allowing semi-quantitative analysis only.

Currently all known approaches for clinical protein detection and quantification involve assays relying on surface immobilisation or bead immobilisation, as for example ELISA assays. While ELISA assays can achieve a relatively high sensitivity, they are not capable of determining biophysical parameters describing the binding interaction in solution, although the physiologically relevant processes take place in solution.

Alternative strategies have emerged for achieving enhanced sensitivity including the use an antibody pair for detecting protein molecules and the use of apparatus developed for flow cytometry for reading out relative intensities. However, these assays are still performed on the surface of a bead and therefore encounter the usual disadvantages of surface-based techniques described above. In addition, these techniques are often time consuming.

Machine learning algorithms have recently been used in protein-protein interactions studies and in particular for the study of protein functions and pathways involved in different biological processes, as well as for understanding the cause and progression of diseases.

Some experimental techniques have been employed for the identification of PPIs but these are limited to a binary output and there is still a gap in identifying, analysing and predicting the biophysical properties in PPIs to provide meaningful outcomes for a patient.

Thus, there is a requirement to provide an in-solution platform that can be used to collate, analyse and recommend outcomes to a patient based on the available biophysical data in the public and measured biophysical properties between biomolecule interactions, such as biomarkers and their specific targets in body fluids in a fully quantitative manner.

Furthermore, understanding the nuances of the increased breadth of biomedical data available is enabling a more personalised approach to disease progression and monitoring so that treatment regimens can be tailored to take into account the patient's individual biomedical data.

It is against this background that the present invention has arisen.

According to the present invention there is provided a system for improving the quantitative analysis of a sample, the system comprising: a device configured to perform quantitative analysis of bio-macromolecular interactions in solution on a fluid sample to provided quantitative analysis data; a data store storing: personal data relating to a plurality of individuals; data relating to bio-macromolecular interactions; processing circuitry configured to access the data store and identify and retrieve data relevant to the sample; set the parameters under which the quantitative analysis of the sample is performed in the device in dependence upon said retrieved data; perform analysis using a general model to create a predicted result of the quantitative analysis from the device; receive quantitative analysis data of the sample from the device; compare said quantitative analysis received from the device with the predicted result; and update said data store with at least one of the output of the comparison and said received quantitative analysis data.

The output of the comparison between the quantitative analysis received from the device and the predicted result may be a confirmation of the predicted result. Conversely, the output of the comparison between the quantitative analysis received from the device and the predicted result may be a deviation from the predicted result.

The circuitry configured to perform said analysis may comprise a machine learning algorithm.

Quantitative analysis of samples enables much greater insight than a mere binary classification of the presence or absence of an indicator. Quantitative analysis introduces a more nuanced diagnostic tool that goes beyond the mere presence or absence of a biomarker.

A data store is an ever-developing repository of information drawn from various sources and involved in all of the intelligence loops. Even if it commences with only low confidence experimental data, this is sufficient to add some value and, as the device undertakes quantitative analysis of samples, the additional data gleaned from this analysis used to augment the knowledge store.

For the system to operate to provide a clinically relevant output, the data store includes personal data relating to a plurality of individuals. This data may include any relevant information from medical records including medication, disease history, disease state and severity, age, gender and weight. Additional data concerning, for example, disease state can be added to the data store when they are computed by the system thereby enabling the patient's disease state to be tracked over time.

A general model relates to, for example, protein-protein interactions themselves. The source of the model may include any experimental or patient data either from the system itself, as proprietary data sets or from third party library data sets. The creation of a model general to the protein-protein interactions themselves also enables missing data to be predicted by interpolation of existing data. The accuracy score of interpolated or predicated data points will reflect the nature of these data points.

In a patient focussed deployment of the system, the sample may be obtained from an individual and the processing circuitry may be configured to perform further analysis of the quantitative analysis data received from the device in order to produce clinically relevant data for the patient.

The clinically relevant data or output may take the form of a binary outcome confirming the presence or absence of a threshold level of a key biomarker, such as, for example, an antibody, in the fluid sample provided. Furthermore, the clinically relevant data may also provide the quantity of the biomarker identified in the sample.

Additionally or alternatively, the clinically relevant output may relate to an incremental change in severity of a previously diagnosed disease state. This may include information about the rate of change of the disease. This information about the disease state can also be combined with the personal data for that individual, which includes information about dosage regimens of medication, in order to provide a clinically relevant output in the form of a recommended dosage modification on the basis of the identified disease severity.

The processing circuitry may further be configured to update the personal data relating to the individual's sample analysed. This provides the closure of the feedback loop at the individual level. The individual's personal data, held within the data store, is augmented with the new quantitative analysis of the sample. This data is stored with the individual's record, along with processed outcomes and other meta-data derived from the quantitative analysis of the sample.

In relation to the data sources deployed with the system, the data relating to bio-macromolecular interactions may include anonymised data from individuals and experimental data.

In the context of the present invention, the term “bio-macromolecular interaction” is used to describe all interactions, either in native form or any chemically modified derivatives thereof, including labelled variants between proteins, peptides, aptamers, nucleic acids and antibodies. Each interaction may be between two bio-macromolecules of the same type or of different types. Protein-protein interactions are bio-macromolecular interactions, as are protein-peptide interactions. The macromolecules may be natural or synthetic. One of the macromolecules may comprise a probe, which may be labelled.

Each data point in the data store may have an associated accuracy score and wherein the step of updating the data store includes updating the accuracy score. The accuracy score will inform the algorithm as to the source of the data, for example, whether the data was obtained using an analogous device to that included in the system or a different device. The accuracy score will further inform the algorithm whether the data correlates with the sample in relation to one or more factors such as the age, gender, weight, disease state, disease severity.

Each predicted result generated by the system may have an associated accuracy score. The accuracy score in the predicted result takes into account both the fundamental or aleatoric error relating to the accuracy of the data and the epistemic error relating to how close the new data point is to the existing model as developed from the training data set.

The data relating to bio-macromolecular interactions may include predicted data based on adjacent data. Where the accuracy score of a subset of the data is sufficiently high, and therefore confidence in the accuracy of that data is sufficiently high, the machine learning algorithm is configured to provide predictions of expected results that lie adjacent in the data space to pre-existing data points with high confidence.

The personal data may include one or more of the following: medical records, age, gender, weight, disease state, disease severity, identity of medication prescribed and corresponding dosage regimen.

The more prior information that can be included in the data store the more accurately the system can analyse a sample from an individual. In general, a subsequent sample, provided for analysis for a given individual can be assessed with better tuned parameters because a more accurate prior is available. The machine learning algorithm can therefore take the personal data from the data store as part of the prior, relying less on data deemed to be analogous from the experimental data on the basis of disease state and severity, for example.

The sample may be a cell culture. Cell culture samples are critically important for informing the general model because they can be less complex as they can be prepared to comprise only the reagents of interest and therefore the quantitative analysis should not be clouded by side reactions or other spurious signals.

The sample may be a bodily fluid. In particular, the sample may be, or may be derived from blood including serum and plasma or it may be CSF, saliva, sweat, faeces or urine.

In relation to the models deployed within the system, wherein the machine learning algorithm may include a plurality of specific models relating to clinically relevant outputs such as disease states. These models can enable a variety of outcomes including a stratification of patients, enabling prediction of disease severity, disease development. These models can also provide a risk assessment as to an individual's risk in relation to a specific disease state.

The machine learning algorithm may be configured such that each quantitative analysis carried out by the device informs both specific and general models. This is the closure of the feedback loop where each quantitative analysis carried out by the system is fed back into the data store and also informs the specific and general models. For example, if quantitative analysis has been carried out in a specific individual's sample, the output of the analysis will be added to that individual's personal data. In addition, the data will be accessible to inform the specific model in relation to other individuals with a similar disease state or severity via an improved specific model of that disease. Furthermore, the data relating to the protein-protein interaction itself will be accessible to the general model.

In relation to the quantitative analysis carried out on the system, the quantitative analysis of the sample may include a measurement of affinity of a bio-macromolecular interaction.

Additionally or alternatively, the quantitative analysis of the sample may include a measurement of the concentration of a bio-macromolecule of interest within the sample.

Additionally or alternatively, the quantitative analysis of the sample includes analysis of the heterogeneity of the sample. The heterogeneity of the sample may include, but is not limited to, the presence of, and/or extent of isoforms, post translational modifications, different stoichiometry, extra binding partners, splice isoforms. The quantitative analysis of the sample may further include analysis of the charge, mobility, hydrodynamic radius, amino acid content within a protein, fluorescence of a protein.

In relation to the parameters set by the machine learning algorithm, these may include sample preparation parameters. The sample preparation parameters may include, but are not limited to, the titration concentration, the buffer concentration and/or the buffer composition such as the pH of the buffer, the identity and concentration of any other chemical components added to the reaction mixture including but not restricted to salts, surfactants, co-solvents and organic molecules, reaction conditions such as time and temperature, any preparation performed on the test sample itself including the status of the same as fresh or frozen; preparatory steps carried out including, but not limited to one or more of filtration, centrifugation, depletion of specific components; and any preparation performed on the added label component including but not limited to one or more of purification, stipulation of storage conditions; the rate of flow and sample and/or the rate of flow of buffer. In an example, an S1 protein, is in complex formation with ACE2 protein in which a titration of serum containing antibodies can be added to the complex. The parameters that can be varied by the machine learning algorithm in this example include: the concentration of the S1 protein, which is unlabelled; the concentration of the ACE2 protein, which is labelled and the concentration, i.e. the extent of titration of the serum. The parameters are varied in order to ensure that the experiment is run under conditions suitable for providing the most rich data output.

Alternatively or additionally, the parameters set by the machine learning algorithm may include device conditions. The device conditions include, but are not limited to, the temperature at which the assay is performed, the voltage applied, the wavelength or wavelengths at which observations take place; anti-adhesion substances that can be used to prevent components adhering to the channel walls of a microfluidic device.

Alternatively or additionally, the parameters set by the machine learning algorithm may include, but are not limited to, the selection of the label such as the type of fluorophore used and therefore the wavelength at which the read out of the data is optimised. Furthermore, the parameters may include one or more additives, such as HSA.

Alternatively or additionally, the parameters set by the machine learning algorithm may include, but are not limited to, setting an expectation of the outcome of the analysis. The setting of an expectation is based on prior information from within the data store. The confidence of the expectation will be affected by the accuracy scores of the relevant data within the data store. When the device subsequently undertakes the quantitative analysis, the analysis may confirm the expectation or it may deviate from the expectation. In the latter case, where the result of the analysis deviates from the expectation, this may result in further quantitative analysis being undertaken which may further inform the models developed by the system.

In relation to the device deployed within the system, the device may comprise a microfluidic network configured to enable combination and distribution of a sample fluid and an auxiliary fluid to create a distributed sample and subsequent division of the distributed sample into two or more parts and measurement of at least one of the parts.

The distribution may be created by one or more of diffusion, electrophoresis or magnetophoresis, thermophoresis, chromatography and isoelectric focusing. Various different chromatographic techniques may also be applicable including, but not limited to size exclusion chromatography and reverse phase chromatography.

The device may be configured to divide the distributed sample into more than two parts and measurement is carried out on each divided part.

The invention will now be described, by way of example only, with reference to the accompanying drawings in which:

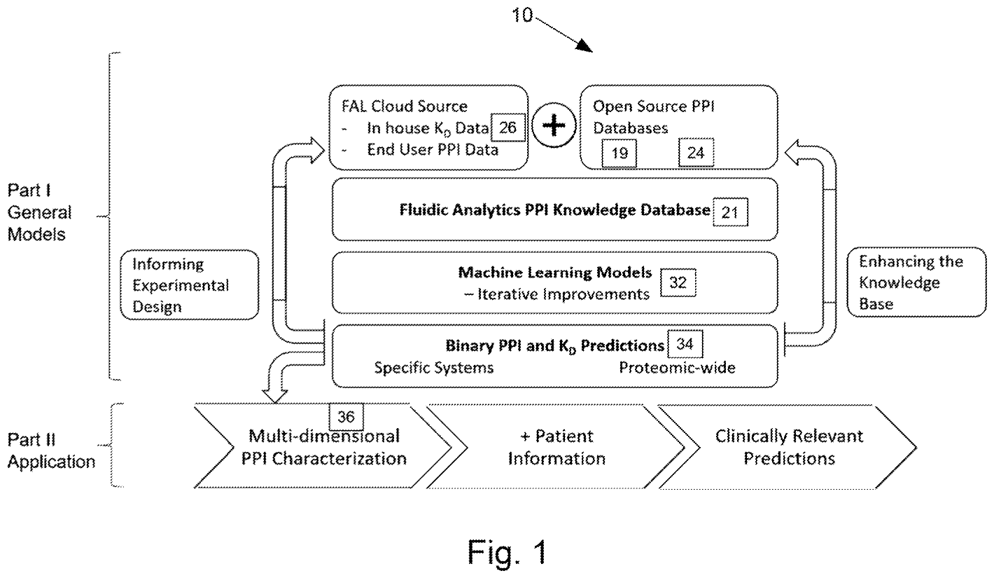

FIG. 1 shows a flow diagram showing the feedback workflows of the present invention which relate to improved experimental design and enhanced data within the data store;

FIG. 1 also shows the application of these feedback workflows to provide clinically relevant predictions;

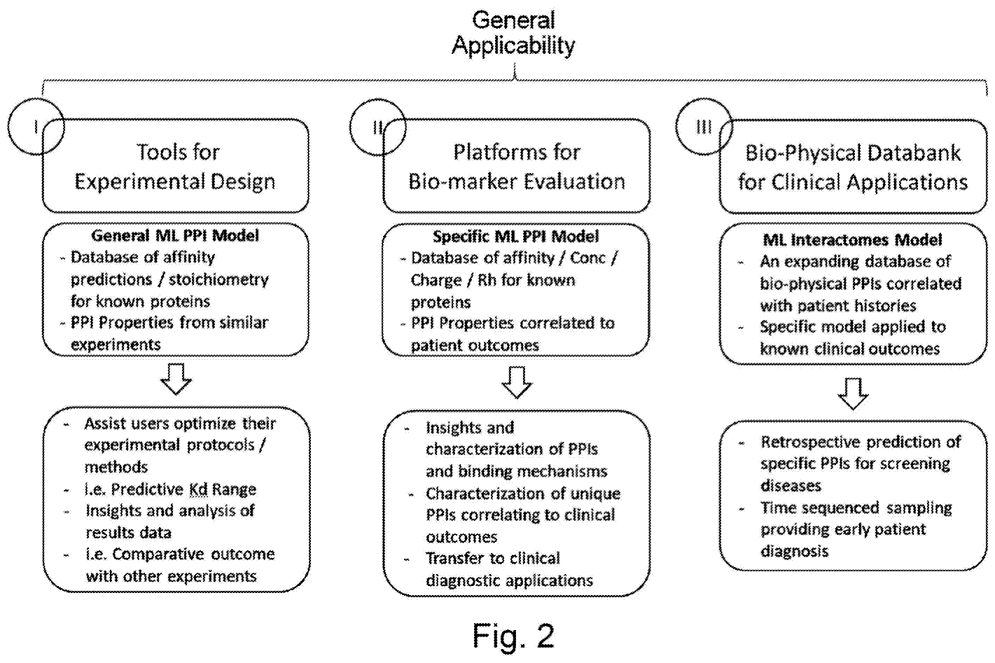

FIG. 2 shows four separate use cases for the system set out in FIG. 1;

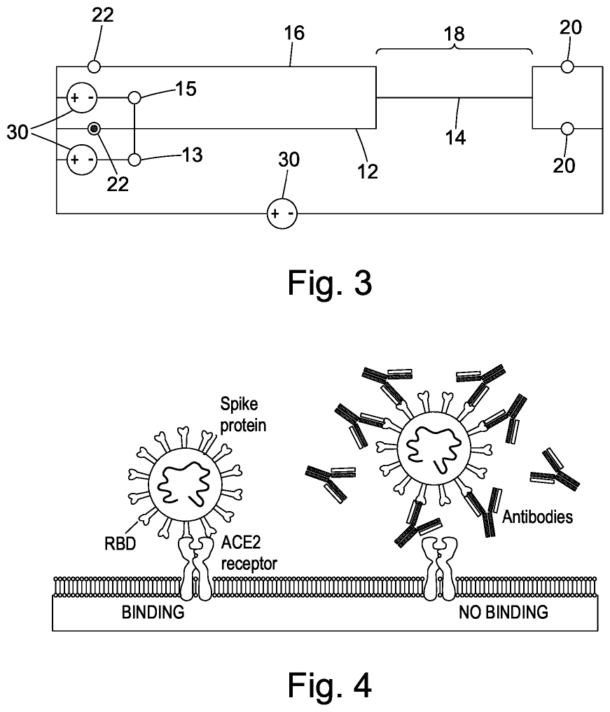

FIG. 3 is a schematic of a microfluidic device provided within the system of the present invention;

FIG. 4 is a schematic of SARS-CoV-2 which is a positive-sense single-stranded RNA virus that is predominantly made up of four main structural proteins: the envelope (E), membrane (M), nucleoprotein (N) and spike (S) proteins;

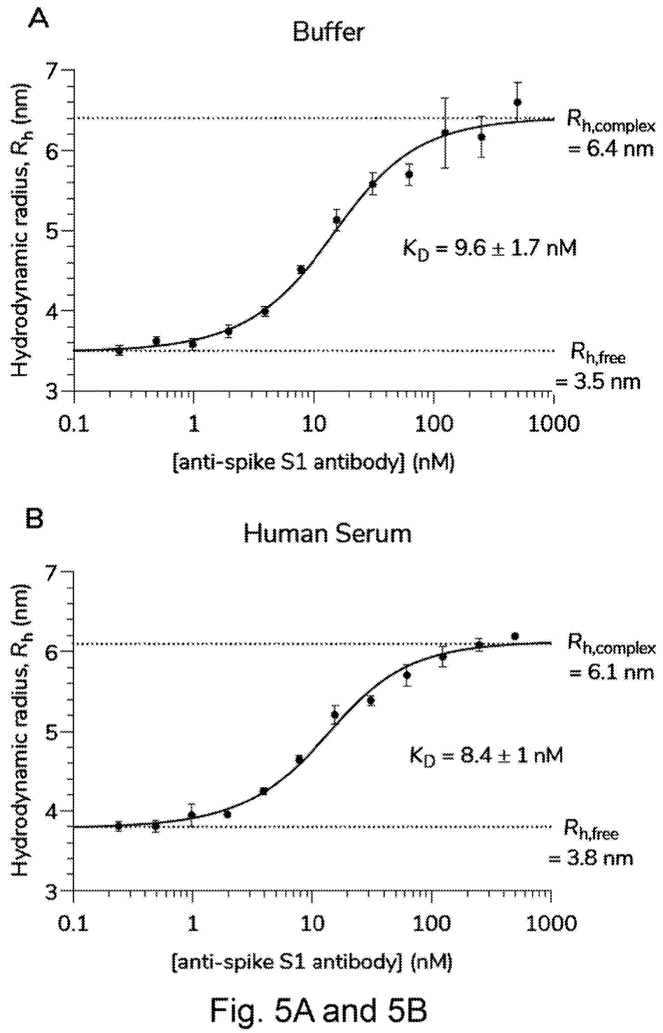

FIG. 5A shows equilibrium binding curves of anti-spike S1 antibody to 20nM Alexa Fluor 647 labelled SARS-CoV-2 RBD in buffer of PBS with 0.05% Tween 20;

FIG. 5B shows equilibrium binding curves of anti-spike S1 antibody to 20nM Alexa Fluor 647 labelled SARS-CoV-2 RBD in human serum;

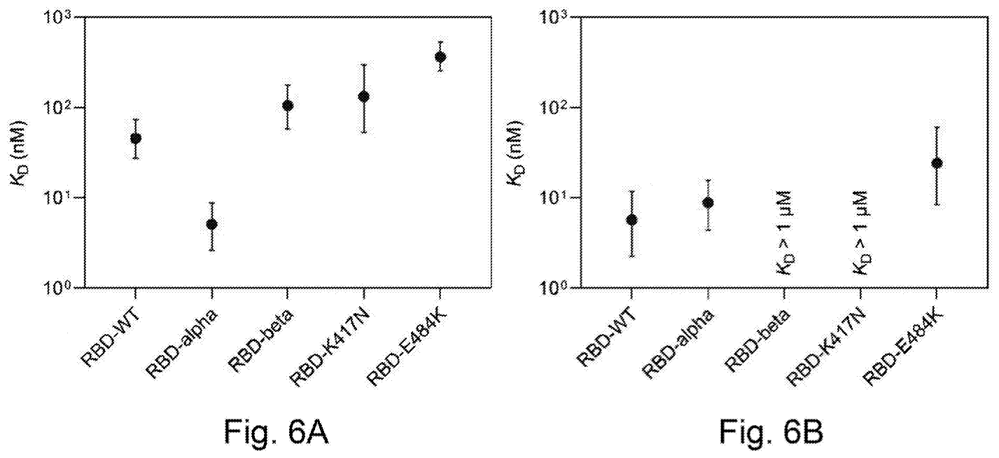

FIG. 6A shows the dissociation constants, K_{D}, of different variants of SARS-CoV-2 RBD binding to human ACE2 receptor;

FIG. 6B shows the dissociation constants, K_{D}, of different variants of SARS-CoV-2 RBD binding to a neutralizing monoclonal antibody; and

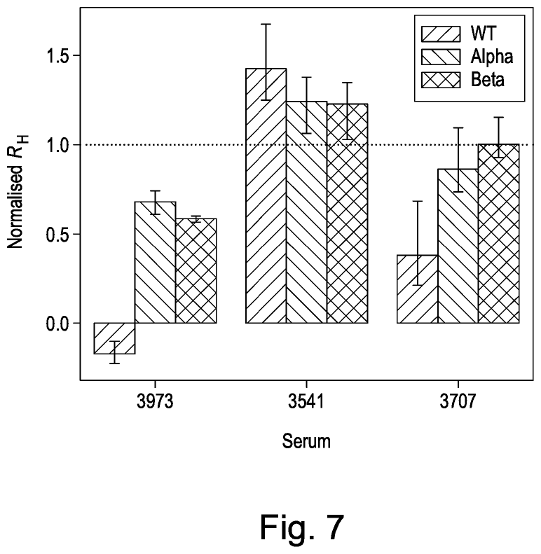

FIG. 7 shows a plot of a receptor-binding competition assay.

FIG. 1 shows the workflow achieved on the system 10 of the present invention. The system 10 includes a data store 19 which can include proprietary knowledge database 21, third party databases 24 including open source general data pertaining to protein-protein interactions or other relevant bio-macromolecular interactions. In addition, the data store 19 includes in house data 26 generated on the device 50 (shown and described in more detail below with reference to FIG. 3). The data store 19 may be a single data store or it may include a plurality of sub stores each storing data from a specific source. These sub stores may be physically co-located with the device 50 or they may be distributed, in particular, cloud based. Regardless of their physical location, they are functionally linked and are therefore referred to generally as the data store 19.

The system 10 also includes a machine learning algorithm 32 which includes at least one general model 34 pertaining to bio-macromolecular interactions. The machine learning algorithm 32 includes a plurality of different algorithms each selected for use with a model. There is at least one general model 34 and one specific model 36 pertaining to a disease state.

When a sample is obtained, whether it arises from a cell culture, antibodies in a buffer, in vitro or in vivo sample, regardless as to source, the protein-protein interaction data pertaining to the interaction to be analysed is obtained from the general model. This general model data is then used to make a prediction of the quantitative result of the analysis of the sample and therefore the most information rich area can be identified. The machine learning algorithm is then used to provide guidance to the device 50 as to the parameters in sample preparation and/or experimental conditions that will allow the quantitative analysis to take place in the information rich zone. The device 50 then undertakes the sample preparation and flows the sample through the device 50, producing the lateral distribution and taking quantitative readings leading to a measurement of, for example, affinity, concentration or heterogeneity of the sample. This measured quantitative data is then compared with the predicted value with one of two outcomes.

If the measured value closely conforms to the predicted value then the model is validated and the sample can be included in the proprietary database with a high accuracy score. Conversely, if the measured value diverges or deviates from the predicted value then this can be indicative that further analysis is required to understand why the divergence has arisen. This iterative approach can lead to further analysis being done on the same sample in order to investigate the divergence between the prediction and the measured data.

When a patient sample is obtained, in addition accessing the general model pertaining to the relevant bio-macromolecular interactions, the machine learning algorithm 32 also accesses the data store 19 to obtain personal data pertaining to the patient. This data may include previous quantitative data from the device 50 from previous quantitative analyses. In addition, this may include patient data such as the patient's age, weight, gender, medication regimen and other relevant risk factors. Furthermore, the machine learning algorithm 32 will access a specific model 36 pertaining to a disease state. The machine learning algorithm 32 will use these three sources of prior to give a predicted quantitative measurement of the bio-macromolecular interaction to be studied within the sample. The machine learning algorithm 32 will also develop and design the experimental conditions under which the device 50 will operate to best observe the predicted quantitative measurement.

Once the device 50 has undertaken the experiments under the conditions dictated by the algorithm 32, the quantitative data is further analysed with reference to the patient data and the specific model to give a clinically relevant output for the patient. This can be a summary of the disease state of the patient including a comparison with previous data to give a rate of advancement of the disease. Furthermore, the clinically relevant output can include recommendations in relation to medicament regimens including alteration of dosage of existing medication or changing of medication utilised.

FIG. 2 shows some of the different modes under which the system 10 can be operated. In an initial, self-referential phase, the system 10 can be operated using samples thereby accessing only the general model and optimising the in house data store or proprietary data store relating to K_{D}, PPI network information, biophysical properties of proteins in solution; hydrodynamic radius, charge, splice- or charge-isoforms through increasing the accuracy score of the data. This will allow the reliance on open source PPI data and public databases to be reduced over time as the locally generated data set increases in size and confidence.

The second mode in which the system 10 of the present invention can be operated is introducing specific models and using the system as a platform for bio-marker evaluation. The device 50 is configured to measure quantitatively the affinity, concentration and/or charge of a bio-macromolecular interaction of interest. This provides insights into the characterisation of protein-protein interactions and binding mechanisms. This, in turn, enables the correlation of protein-protein interactions to clinical outcomes. This allows the retrospective prediction of specific PPIs for screening diseases. Furthermore, time sequenced sampling provides early diagnosis for individual patients.

A third mode in which the system 10 of the present invention can be operated incorporates a protein fingerprint approach combining a plurality of specific model and other probe facilitated analysis with a probe free approach to determine the biophysical properties between bio-macromolecular interactions. Hypothesis free data acquisition mode where measuring the biophysical properties that the device is configured to measure and correlating that data in combination with patient data to a clinical outcome.

A probe can be used for attachment onto a specific sequence at a binding site or onto a specific surface of a biomolecule of interest. The probe can be labelled, for example with a fluorophore, in order to enable a user to visualise and detect the bio-macromolecule to be quantified. Examples of suitable fluorophores are dyes of the Alexa Fluor™, ATTO, DyLight or other families exemplified by, but not limited to, individual dyes such as DyLight 350, ATTO 488, DY-489XL, Alexa Fluor™ 647 or Alexa Fluor™ 700. Dyes are not restricted to visible wavelength fluorescence, and may be active in the UV, visible or IR regions of the spectrum.

A probe with a label such as a fluorescence label may be desirable because it can provide more flexibility in choosing the location of the label and the enhanced fluorescence properties can be suitable for a greater number of biophysical techniques used for quantitative analysis of the biomolecule of interest. Therefore, providing a probe with a label attached to the probe within the system of the present invention can be highly advantageous as the probe can enable a user to determine accurately and quickly one or more biophysical properties of the bio-macromolecular interactions such as the affinity, concentration and/or charge of a bio-macromolecular interaction of interest.

In some instances, an example of a probe-free approach may also be deployed in which bulk labelling of one or more residues exposed on the surface of a biomolecule of interest can be used to help determine one or more biophysical properties of the bio-macromolecular interactions, such as the affinity, concentration and/or charge.

Alternatively or additionally, another example of probe-free approach may be to utilise the intrinsic fluorescence of a biomolecule such as detecting the intrinsic fluorescence from the aromatic residues of a protein at a specific wavelength. Utilising the intrinsic fluorescence properties of a biomolecule can provide information on the biophysical properties of a bio-macromolecular interaction in its native state and therefore, this probe-free approach can be highly advantageous as it does not require a probe which can often distort the natural binding or interactions between molecules.

Other examples of biophysical properties of the biomolecular interactions that can be measured using the device 50 include hydrodynamic radius, from which the molecular weight can be inferred using experimental data to make this inference; mobility from which charge can be inferred; hydrophobicity, acid content via labelling or intrinsic fluorescence, pl is via isoelectric focusing, Trp and Tyr via UV intrinsic fluorescence, or labelling of the specific amino acid residues such as Met, Lys and/or Cys residues with fluorophores.

### Machine Learning Algorithm

The term machine learning algorithm is used to refer generally to a combination of numerous different algorithms each of which is selected for use with a respective aspect of the experimental design and/or clinical output aspect. Different algorithms will be appropriate for general models of bio-macromolecular interactions, for specific models of disease progression and for affinity measurement.

For example, for the general model a fully connected deep neural network, recurrent neural network, convolutional neural network or self-attention based architectures, such as transformer based architectures, may be deployed. Representational learning may be used to generate embeddings of sequences and structures of biomacromolecules. These algorithms may be combined with classifier or regressor systems as appropriate. Examples of classifiers that may be appropriate for a general include random forests, gradient boosting machines, Gaussian processes or multilayer perceptrons. A single classifier may be deployed. However, in some embodiments the stacking of classifiers may be achieved. In order to achieve an effective stacking of classifiers, the classifiers are trained to predict the error in the output, rather than the output itself. A combination of Gaussian process and multilayer perceptrons is effective in this context.

The stacking of classifiers is advantageous in this context because the field of bio-macromolecular interactions is complex and the data sets are comparatively small.

In order to introduce the data into the machine learning algorithm it is first necessary to vectorise the bio-macromolecule of interest, for example, a protein, so that the complex structure of the protein can be represented as a numerical vector. This vector can then be ingested by the classifier and thus processed through the machine learning algorithm. This allows the data to be used, initially, to train the machine learning algorithm and, in combination with many other similarly vectorised bio-macromolecular data, to develop a prediction of new quantitative analysis of the interaction of that bio-macromolecule.

The specific models, relating to the modelling of disease progression and disease state, a tabular data set with associated data transformation and encoding for algorithms can be deployed. Similar classifiers or regressors as described above with reference to the general model may be deployed. In addition, specific models will be informed additionally by information from the general model.

FIG. 3 shows an example of a device 50 that can be incorporated into the system 10. FIG. 3 shows a device 50 configured to provide separation and analysis of a plurality of components in a heterogeneous sample. The device incorporates two sections: a capillary electrophoresis section and an H-filter 18. Although, in the illustrated embodiment, the capillary electrophoresis section precedes the H-filter, it will be appreciated, that the order can be switched so that the H-filter is deployed first. In that configuration, a capillary electrophoresis module can be applied to each of the outputs of the H-filter so that there are as many capillary electrophoresis modules as there are outputs of the H-filter.

The component may be a biological and/or chemical component, or it can be a biomolecule. The biomolecule can be, but is not limited to a protein, a peptide, polysaccharide, nucleic acid such as DNA, RNA, an antibody or an antibody fragment thereof.

The device 50 includes the constituent parts of an H-filter 18 with a sample channel 12 and a buffer channel 16 through which the sample and a buffer or auxiliary fluid can be introduced. The sample channel 12 and the buffer channel 16 terminate at a distribution channel 14 that is elongate is a first direction.

As a result of the elongate configuration of the channels, when a sample flows along the sample channel 12, into and through the distribution channel 14, a distribution of the components in a second direction, substantially perpendicular to the first direction will develop.

The device 50 may include at least one power source 30 configured to provide an electrical field across the distribution channel 14 of the H-filter 18 in order to drive the distribution by electrophoresis.

The H-filter 18 has two outlets 20 and the fluid in the distribution channel 14 is divided between the two outlets. Quantitative analysis of the fluid collected at each of the outlets can be undertaken and data can be compared between the outlets 20. The quantitative analysis will be associated with the regimen under which the lateral distribution was created. Therefore in the device illustrated in FIG. 3, where the power source 30 creates an electrical field across the distribution channel 14 so that the distribution is achieved through electrophoresis, then the quantitative analysis is of the charge on the components within the sample.

Conversely, if the power source 30 in FIG. 3 is not activated, then the distribution in the distribution channel 14 will arise solely via diffusion and the quantitative analysis will be related to the size of the components within the sample.

Alternatively, or additionally, the distribution created in the distribution channel may be achieved by capillary electrophoresis. In addition, the lateral distribution can be created diffusively, electrophoretically, diffusophoretically, magnetophoretically or thermophoretically.

The device 50 is configured to separate and analyse fluid samples using capillary electrophoresis (CE) separation and diffusive sizing. As shown in FIG. 3, the device 50 comprises an H-Filter 18 with one or more extended inlets 22. Loading of the sample takes place through a sample port 13 into the separation channel 12 and is either achieved via electro-osmotic flow (EOF) or it is pressure-driven. Once the sample has reached the separation channel 12, an electric field is applied across both ends i.e. inlets 22 and outlets 20 of the H-filter 18 to drive the entire distribution channel 14 electro-osmotically. In order to provide control over the sample being supplied to the separation channel 12 there is a sample waste port 15 corresponding to the sample inlet port 13.

As shown in FIG. 3, there is provided at least one power source 30 so that a voltage can be applied to the separation channel 12 and the auxiliary channel 16. FIG. 3 shows exemplary configurations for the voltage supplies 30 and electric connections that can be used to run the device 50 of the present invention. The appropriate selection of the polarity of the power supply 30 will depend on the predicted charge on the components in the sample.

In the embodiment illustrated in FIG. 3, the separation channel 12 and the auxiliary channel 16 are of equal length. The separation channel 12 and the auxiliary channel 16 also have equal cross sectional area. Having the separation channel and the auxiliary channel of equal dimensions simplifies the control of the electro-osmotic flow through the device as the same voltage can be applied across both the separation channel 12 and the auxiliary channel 16, thereby providing substantially equal electro-osmotic flow rates in the separation channel 12 and the auxiliary channel 16.

Moreover, the symmetry between the auxiliary channel 16 and the separation channel 12 ensures equal flow entering the distribution channel 14 and/or throughout the whole H-filter 18. Flow sensors or reference samples (not shown in the accompanied Figures) can be included to determine the bulk flow rate. Reference samples can be introduced into either the separation channel or the auxiliary channel.

Furthermore, the sample can be separated via CE in the separation channel 12 and then can be subjected to diffusive sizing in the H-filter 18. The symmetry the separation channel 12 and the auxiliary channel 16, as well as the constant applied electric field across both channels may provide well-defined flow rates. In some embodiments, the auxiliary capillary may also contain a cross-channel (not shown in the accompanied Figures) for sample loading to enhance symmetry.

The device 50 may also include a sample preparation module (not shown in the accompanying Figures) in which the sample can be prepared ready for introduction into the sample channel 12. The sample preparation module includes a microtitrator to enable the concentration of the sample to be controlled. The sample preparation module also includes temperature and humidity controlled storage conditions so that the sample preparation module can mix and store the sample under conditions stipulated by the machine learning algorithm for a time period recommended by the machine learning algorithm. The mixture created in the sample preparation module may include ternary or higher order mixtures.

### Detailed Description of One Exemplary Quantitative Analysis

Quantitative measurement of affinity, sometimes also referred to as the dissociation constant or K_{D}, under physiologically relevant conditions in complex mixtures like serum can provide useful insight into immune response and protection window in patients and vaccinated individuals.

Accurate affinity profiling of a SARS-CoV-2 antibody in serum can be undertaken in the device of FIG. 3 using microfluidic diffusional sizing to characterise an anti-spike S1 antibody by measuring its binding affinity to the receptor binding domain (RBD) of the SARS-CoV-2 spike protein in serum.

In order to accurately identify individuals who are seropositive as well as finding the most effective vaccines against the recently emerged coronavirus SARS-CoV-2, it is fundamental to thoroughly characterize the immune response in the course of the infection or after vaccination. This includes both a binary indication as to whether or not the individual has been exposed to the virus and also a quantitative assessment as to the level of antibodies present in a patient sample. In particular, the virus—neutralizing capacity of the immune system is of vital interest, and accurate tests to evaluate the affinity and quantity of neutralizing antibodies (NAbs) in serum samples of COVID-19 patients or vaccinated individuals are key.

As illustrated schematically in FIG. 4, SARS-CoV-2 is a positive-sense single-stranded RNA virus that is predominantly made up of four main structural proteins: the envelope (E), membrane (M), nucleoprotein (N) and spike (S) proteins. The spike protein is crucial for virus entry into the host cell. It is composed of two subunits: S1, which binds to the host cell receptor ACE2; and S2, which mediates the subsequent fusion of the virus with the cell membrane.

Due to its key role mediating the first step of viral invasion of host cells, the RBD (receptor binding domain) of S1 has proven to be the target of neutralising antibodies raised against other viruses of the corona family, and is an important target in the case of SARS-CoV-2.

The device shown in FIG. 3 enables the concentration and affinity of the antibody to be simultaneously and independently determined. This aids understanding of immune response. This in turn allows a better understanding of antibody maturation and persistence of immunity and, in the future, could aid in convalescent plasma therapy research and vaccine design.

Measuring antibody affinity in human samples ideally makes use of undiluted serum to maximize the range of antibody concentrations that can be used to generate the equilibrium binding curve. Most established technologies for measuring protein binding, however, rely on surface immobilization of one of the binding partners. This can cause significant difficulties when working with complex samples such as serum due to non-specific binding of other proteins within the serum to the analytical surface, leading to false positives or at least low signal-to-noise ratios.

In the device shown in FIG. 3, microfluidic diffusional sizing is used to measure the affinity of an anti-spike S1 antibody to fluorescently labelled SARS-CoV-2 RBD directly in serum. This in-solution technology enables the detection of antigen—antibody interactions by measuring the changes in hydrodynamic radius (Rh) of the labelled antigen upon binding to the antibody. As a result, MDS allows the accurate detection and characterization of antibodies directly in serum, thus eliminating the constraints of surface-bound technologies.

### EXAMPLE

SARS-CoV-2 RBD (40592-VO8H, Sino Biological) was reconstituted in 400 μL sterile water to a concentration of 0.25 mg/mL. For labelling, the protein was diluted into labeling buffer (0.2 M NaHCO3 pH 8.3) and mixed with Alexa Fluor™ 647 NHS ester (Thermo Fisher Scientific at a dye-to-protein ratio of 10:1. Following incubation overnight at 4° C., labelled RBD was purified via size exclusion chromatography using a Superdex 75 Increase 10/300 GL column with PBS (pH7.4) as elution buffer.

For affinity measurements in serum, SARS-CoV-2 (2019-nCoV) spike antibody (40150-R007, Sino Biological) was diluted in human serum (H5667, Sigma), to achieve a two-fold concentration series ranging from 490 pM to 1 uM. Antibody dilutions were subsequently mixed in a 1:1 ratio with a 40 nM solution of Alexa Fluor 647 labelled SARS-CoV-2 RBD, to obtain a final IRBD concentration of 20 nM. All samples were incubated for 30 min at 4° C. prior to measurement and kept at 4° C. throughout the experiment.

For affinity measurements in buffer, antibody and Alexa Fluor 647 labelled protein were diluted in PBS with 0.05% Tween 20 instead of serum. Concentrations and incubation times were identical to experiments in serum.

Samples were measured on the device of FIG. 3 using a 1.5-8 nm size-range setting. Measurements were performed in triplicate at room temperature. To correct for background fluorescence caused by serum, independent measurements of human serum were performed, and a background subtraction was applied to individual data points obtained in serum. The binding affinity K_{D}, was automatically generated by non-linear least squares fitting to Equation 1.

$${R}_{h} = {R}_{h , free} + ( {R}_{h , complex} - {R}_{h , free} ) \times \frac{\begin{matrix}{[ U ]}_{tot} + {n [ L ]}_{tot} + {K}_{D} - \\ \sqrt{{( {[ U ]}_{tot} + {n [ L ]}_{tot} + {K}_{D} )}^{2} - 4 {{n [ L ]}_{tot} [ U ]}_{tot}}\end{matrix}}{2 {n [ L ]}_{tot}} \qquad Equation 1$$

Where:
Rₕ is the hydrodynamic radius at equilibrium
R_{h,free} is the hydrodynamic radius of the unbound protein
R_{h,complex} is the hydrodynamic radius of the protein-ligand complex
[L]ₜₒₜ is the total concentration of labeled species
[U]ₜₒₜ is the total concentration of unlabeled species
n is the complex stoichiometry (unlabeled molecules per labeled molecule fixed at 0.5
K_{D} is the dissociation constant

Since absolute sizes indicated that two molecules of SARS-CoV-2 RBD bound to one antibody, the stoichiometric parameter, n, was set to 0.5.

To assess the binding affinity of the anti-spike S1 antibody to the RBD of the SARS-CoV-2 spike protein, the antibody was titrated against a constant concentration of 20 nM Alexa Fluor 647 labelled recombinantly expressed RBD. As a control, the titration experiment was first performed in buffer. FIG. 5A shows the affinity binding curve measured in PBS with 0.05% Tween 20, yielding a KD of 9.6±1.7 nM.

For the measurements in human serum, the same antibody concentrations were titrated against 20 nM Alexa Fluor 647 labelled RBD, with the antibody diluted in serum. In relation to FIG. 5B, which shows serum measurements, serum background fluorescence was subtracted from raw data before the K_{D} was determined by non-liner least squares fitting using Equation 1 above. Dependent on the dilution factor of the unlabelled anti-spike S1 antibody, the respective serum concentrations ranged from 91-97%, and, despite the high concentrations of serum in these samples, the K_{D} value determined for this interaction matches that in PBS.

This example shows a single quantitative analysis performed using MDS on the device of FIG. 3 to accurately detect and characterize the binding affinity of antibodies to virus proteins directly in human serum. Thus, this technology could be used for in-depth analysis of the humoral immune response against SARS-CoV-2 to support the development of reliable antibody tests and vaccines in the fight against the COVID-19 pandemic.

Each quantitative analysis is then added to the data store alongside the personal data of the patient including associated risk factors such as patient weight, age, other unrelated diagnoses such as heart disease, asthma etc. As more analyses are collected the system is able to more accurately predict the immune response of another patient on the basis of the quantitative analyses collected to date.

The above described example the S1 spike protein effectively acts as a probe. However, the system can also be used as a probe free system in solution. An example of a probe free system can require bulk labelling of the surface exposed residues such as lysine residues to identify and detect bio-macromolecular interactions. Lysine residues are very frequent in proteins and therefore labeling the exposed lysine residues on the surface of a protein can help detect and visualize the binding of another biomolecule on the surface of the labelled protein. However, it may be possible to specifically target the N-terminal α-amino group which may facilitates successful labeling at a specific, but limited location on the surface of the biomolecule.

In this example, the label does not affect the separation, unlike, for example, the use of magnetic labels followed by the application of a magnetic field to the distribution channel so that the distribution is predicated on the label. However, the probe may contribute to the distribution depending on the regimen under which the distribution is created. For example, if the distribution is created by diffusion then the mass of the probe will contribute to the diffusion of the bio-macromolecule through the distribution channel. Similarly, if the distribution is created electrophoretically then the charge of the probe will contribute to the creation of the distribution.

Another example of a probe free approach requires the detection and/or measurement of the intrinsic fluorescence properties of a biomolecule, such as an amino acid. For example, the aromatic residues of a biomolecule of interest such as tryptophan, phenylalanine and/or tyrosine residues can be excited at a specific wavelength and the excited aromatic residues can emit fluorescence at a different wavelength which can be detected using a U.V/fluorescence spectrometer. Thus, the biophysical properties such as the affinity, concentration, molecular weight, amino acid contents between bio-macromolecular interactions can be measured based on its intrinsic fluorescence properties.

Probe based and probe free datasets can be combined in a protein fingerprint. A protein fingerprint combines all possible data available, each data point having its own accuracy score to enable confidence in the veracity of that data point to be ascertained. The data is aggregated from various sources and is augmented over time allowing the evolution of the well-being or tracking of disease states to be undertaken. Furthermore, as specific models are further developed and new specific models are created, the data within the protein fingerprint can be interrogated again and again. This enables new findings to be made within pre-existing data. For example, diagnosing a new condition based on previously obtained data.

The system of the present invention as disclosed herein can be elaborated in a further example below. The example as described below aims to investigate the different strategies through which SARS-CoV-2 variants, such as Alpha and Beta variants, are capable of antibody escape. Variants that are capable of antibody escape can lead to both higher transmission and more symptomatic disease in those infected.

As used herein, and unless otherwise specified, the term “antibody escape” means that the mutations of the virus, which can occur randomly, initiate a change in the structure of the antigen present on the surface of the virus and thus, making the antigen unrecognisable by antibodies that were developed against a previous infection by an unmutated strain of the same virus.

As shown in FIG. 1, the proprietary knowledge database 21, which is also referred to as the PPI knowledge database, contains binding affinity information about specific protein-protein interactions. For example, SARS-CoV-2Receptor Binding Domain (RBD) of the S1 protein with a potent neutralising monoclonal antibody SAD-S35 and SARS-CoV-2 S1RBD with human ACE2 receptor. This information can be obtained via a variety of sources. In one example, the data can be obtained with reference to external open source databases 19, 24, as well as through in-house determination of binding affinity 26, which can be generated on the device 50 as disclosed in the present invention. A comparison step between the data obtained by external open source databases 19, 24 and the data obtained through in-house 26 by the device 50 shows that they are in agreement. The in-house measured values are plotted with uncertainty in FIGS. 6A and 6B, for RBD-WT (wild-type) on the x-axis.

FIGS. 6A and 6B show the dissociation constants, K_{D}, of different variants of SARS-CoV-2 RBD binding to the ACE2 receptor (FIG. 6A) and to a neutralising monoclonal antibody (FIG. 6B). The equilibrium binding can be measured by microfluidic diffusional sizing (MDS) for various concentrations of fluorescently labeled RBD variants and unlabeled ACE2 or unlabeled neutralising antibodies (Nab). The K_{D} values can be determined from modes of posterior probability distributions obtained by Bayesian inference of the kinetic equilibrium model that describes the binding interaction. Error bars are 95% credible intervals, as shown in FIGS. 6A and 6B.

As illustrated in FIGS. 6A and 6B, the binding affinities refer to the wild-type (or Wuhan strain) of SARS-CoV-2. The PPI knowledge database 21, as shown in FIG. 1, contains further information about Variants of Concern (VoCs) of SARS-CoV-2. Based on this information, a set of mutant S1-RBD proteins can be used and experimental parameters set for determining the binding affinities of these mutants to the ACE2 receptor and the neutralising antibody SAD-S35.

The binding affinity of mutant S1-RBD proteins to both targets can be measured using the device 50 as disclosed herein. The results are shown in FIG. 6. Importantly, RBD-alpha shows increased affinity (by a factor of 10) for the ACE2 receptor, but its affinity for the neutralising antibody SAD-S35 is largely unaffected. Conversely, RBD-beta is not bound by the neutralising antibody SAD-S35, while its affinity for the ACE2 receptor remained the same.

Furthermore, measuring individual mutants can discern the effect of K417N as the mutation that is responsible for antibody escape. The effect of introducing K417N alone demonstrates the mechanism by which the Beta variant achieves antibody escape. Of the several mutations in Beta, K417N is the mutation that is responsible for remodelling the epitope that is recognised by the neutralising antibody.

The results obtained by measuring the above-described affinities in vitro are fed back into the PPI knowledge database 21 and then entered into the machine learning algorithm 32 for the specific system of competition between ACE2 and neutralising antibodies for binding to S1-RBD and antibody escape 32, 34. In combination with prior knowledge about the properties of antibody response profile against SARS-CoV-2 S1-RBD in seroconverted patients, the system can be used to predict the effects of S1-RBD mutations on the antibody profile against it in patients with immunity derived from infection with the wild-type strain. Specifically, the system predicted lower virus neutralising capacity against both Alpha and Beta VoCs, although the dominant effect is different between the two cases.

The predictions are then confirmed through an in-solution receptor binding competition assay on the device 50, which measures the neutralising capacity of patient serum antibodies against the S1 protein by displacing it from the ACE2 receptor. The results are shown in FIG. 7.

Referring to FIG. 7, there is shown an in-solution receptor-binding competition assay. The assay, as shown in FIG. 7, illustrates the size of the SARS-CoV-2 complex in which the labelled ACE2 receptor is found. If it is bound by S1, the size is larger (Rh=1, normalised). If antibodies outcompete S1, the ACE2 is free of complex and is therefore smaller (Rh=0, normalised). So, the assay can show if neutralisation occurs. Error bars are standard deviations obtained from triplicate measurements.

As illustrated in FIG. 7, two of the three patients (3973 and 3707) showed lower neutralising capacity against both VoCs than the wild-type strain, confirming the earlier predictions. Patient 3541 did not have neutralising antibodies against any of the variants.

Using the PPI knowledge database 21 together with the data obtained from the device 50, the results provide a mechanistic hypothesis explaining the different antibody escape mechanisms employed by the two VoCs. Alpha binds the ACE2 receptor with stronger affinity, making it more difficult for a neutralising antibody to displace it. Conversely, mutations such as K417N in Beta remodel the epitopes used by neutralising antibodies raised against the wild-type and prevent their recognition of and binding to the antigen. In both cases, this leads to a higher chance of infection.

To conclude, the system comprising information feedback loops as illustrated in FIG. 1, in combination with measurements using the device 50, is able to characterise a complex protein-protein interaction and make mechanistically interpretable and clinically relevant predictions, which can be validated on patient samples.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A system for improving the quantitative analysis of a sample, the system comprising:
a device configured to perform quantitative analysis of bio-macromolecular interactions in solution on a fluid sample to provided quantitative analysis data;
a data store storing:
personal data relating to a plurality of individuals;
data relating to bio-macromolecular interactions;
processing circuitry configured to
access the data store and identify and retrieve data relevant to the sample;
set the parameters under which the quantitative analysis of the sample is performed in the device in dependence upon said retrieved data;
perform analysis using a general model to create a predicted result of the quantitative analysis from the device;
receive quantitative analysis data of the sample from the device;
compare said quantitative analysis received from the device with the predicted result; and
update said data store with at least one of the output of the comparison and said received quantitative analysis data.

2. The system according to claim 1, wherein the output of the comparison between the quantitative analysis received from the device and the predicted result is a confirmation of the predicted result.

3. The system according to claim 1, wherein the output of the comparison between the quantitative analysis received from the device and the predicted result is a deviation from the predicted result.

4. The system according to claim 1, wherein circuitry configured to perform said analysis comprises a machine learning algorithm.

5. The system according to claim 1, wherein the sample is obtained from an individual and wherein the processing circuitry is configured to perform further analysis of the quantitative analysis data received from the device in order to produce clinically relevant data for the patient.

6. The system according to claim 5, wherein the processing circuitry is further configured to update the personal data relating to the individual's sample analysed.

7. The system according to claim 1, wherein the data relating to bio-macromolecular interactions includes anonymised data from individuals and experimental data.

8. The system according to claim 1, wherein each data point in the data store has an associated accuracy score and wherein the step of updating the data store includes updating the accuracy score.

9. The system according to claim 1, wherein each predicted result generated by the system has an associated accuracy score.

10. The system according to claim 1, wherein the data relating to bio-macromolecular interactions includes predicted data based on adjacent data.

11-13. (canceled)

14. The system according to claim 4, wherein the machine learning algorithm includes a plurality of specific models relating to clinically relevant outputs such as disease states.

15. The system according to claim 4, wherein the machine learning algorithm is configured such that each quantitative analysis carried out by the device informs both specific and general models.

16. The system according to claim 1, wherein the quantitative analysis of the sample includes a measurement of affinity of a bio-macromolecular interaction.

17. The system according to claim 1, wherein the quantitative analysis of the sample includes a measurement of the concentration of a bio-macromolecule of interest within the sample.

18. The system according to claim 1, wherein the quantitative analysis of the sample includes analysis of the heterogeneity of the sample.

19. The system according to claim 4, wherein the parameters set by the machine learning algorithm include sample preparation parameters.

20. The system according to claim 4, wherein the parameters set by the machine learning algorithm include device conditions.

21. The system according to claim 4, wherein the parameters set by the machine learning algorithm include setting an expectation of the outcome of the analysis.

22. The system according to claim 1, wherein the device comprises a microfluidic network configured to enable combination and distribution of a sample fluid and an auxiliary fluid to create a distributed sample and subsequent division of the distributed sample into two or more parts and measurement of at least one of the parts.

23. (canceled)

24. The system according to claim 1, wherein the device is configured to divide the distributed sample into more than two parts and measurement is carried out on each divided part.
